(19) United States
(12) Patent Application Publication
(10) Pub. No.: US 2013/0245465 A1
(43) Pub. Date: Sep. 19, 2013

(54) SLEEP DEPTH DETERMINING APPARATUS AND METHOD

(71) Applicant: FUJITSU LIMITED, Kawasaki-shi (JP)

(72) Inventor: Kouichirou KASAMA, Kawasaki (JP)

(73) Assignee: FUJITSU LIMITED, Kawasaki-shi (JP)

(21) Appl. No.: 13/803,823

(22) Filed: Mar. 14, 2013

(30) Foreign Application Priority Data
Mar. 16, 2012 (JP) 2012-060392

(51) Int. Cl. A61B 5/00; A61B 5/11; A61B 5/0205

(52) U.S. Cl. CPC A61B 5/4812; A61B 5/02055; A61B 5/11
USPC 600/483

(57) ABSTRACT

There is provided an apparatus for determining depth of sleep. The apparatus includes a heart rate sensor configured to detect a heart rate of a subject in a non-contact manner, and a processor. The processor performs determining depth of sleep of the subject from the heart rate detected by the heart rate sensor, estimating a sleep cycle of the subject on the basis of a plurality of times at each of which a change in the depth of sleep occurs, and stopping the heart rate sensor, after completion of acquiring the plurality of times, at least until an anticipated time of a change in the depth of sleep.

### CROSS-REFERENCE TO RELATED APPLICATION

This application is based upon and claims the benefit of priority of the prior Japanese Patent Application No. 2012-060392, filed on Mar. 16, 2012, the entire contents of which are incorporated herein by reference.

### FIELD

The embodiments discussed herein are related to an apparatus and method for determining depth of sleep.

### BACKGROUND

A method of determining depth of sleep from the ratio of a low frequency component to a high frequency component of the fluctuation in heart rate is known. A system for measuring, analyzing, and displaying one or more of a respiration parameter, cardiac activity, and bodily movement or function of a subject is also known. This system includes a transmitter arrangement configured to propagate a radio frequency signal toward the subject, a receiver arranged to receive a radio-frequency signal reflected from the subject, a processor arranged to analyze the reflected signal to produce measurements of one or more of a respiration parameter, cardiac activity, and a bodily movement or function, and a monitor to provide selected information to a local or remote user of the system by either an audible or visual indication, or both.

A behavior recognizing device including a body movement measuring unit to measure body movement, a physiological quantity measuring unit to measure physiological quantities, and a behavior determining unit to determine a behavior type on the basis of body movement measured by the body movement measuring unit and physiological quantities measured by the physiological quantity measuring unit is also known. The body movement measuring unit includes an acceleration sensor, the physiological quantity measuring unit includes a pulse sensor to measure a pulse rate and a temperature sensor to measure body temperature, and the behavior determining unit determines a behavior type on the basis of outputs of the sensors. Examples of the behavior type include movement, sleep, and eating and drinking. The behavior determining unit determines that the behavior type is sleep if the output of the pulse sensor is smaller than a first threshold and the output of the temperature sensor is smaller than a second threshold.

A sleep state determining device including a body movement detecting unit to detect body movement of a human body, and a sleep state estimating unit to estimate a sleep state on the basis of outputs of the body movement detecting unit is known. The sleep state estimating unit includes a neural network model unit having therein a plurality of fixed coupling weight coefficients of a neural network for estimating a sleep state. The fixed coupling weight coefficients are acquired by a method of simulating a hierarchical neural network in which many layers made up of a plurality of nerve elements are combined. The sleep state estimating unit estimates a sleep state on the basis of both outputs of the body movement detecting unit and a skin temperature sensor that detects skin temperature of a sleeper. The sleep state estimating unit estimates a sleep state on the basis of both outputs of the body movement detecting unit and the heart rate computing unit that computes a heart rate on the basis of the output of the body movement detecting unit.

A sleep determination method for determining whether a person is awake or asleep is also known. The sleep determination method includes an active mass detecting process of detecting the active mass of a human body, a body temperature detecting process of detecting the temperature of the person, and an eyelid detecting process of detecting eyelid-state information on whether eyelids of the person are open or closed. The sleep determination method includes an information storing process of storing, in a storage unit, the active mass detected in the active mass detecting process, the temperature detected in the body temperature detecting process, and the eyelid-state information detected in the eyelid detecting process. The sleep determination method includes a sleep determining process of determining, on the basis of the active mass, the temperature, and the eyelid-state information stored in the storage unit, whether the person is awake or asleep.

A biological data analyzer is also known. The biological data analyzer includes a biological data detecting unit and an analyzing unit. The biological data detecting unit converts biological data of a living body during sleep into numbers which is detected as biological data values, and the analyzing unit determines a sleep state, or predicts the physical condition of the living body after sleep, on the basis of the temporal progression of the biological data values during sleep. The biological data is one of a pulse, blood pressure, body temperature and breathing, and the corresponding biological data value is one of a pulse rate, a blood-pressure value, a body temperature value, and a breathing rate.

As a device that measures the sleep-wake rhythm, the actigraph (product name) of Ambulatory Monitoring Inc. (A.M.I.), for example, is known. One one-way vibration sensor that may detect a very fine vibration is mounted in the actigraph, and therefore the actigraph calculates the body movements of a subject at arbitrary time intervals using the number of times an acceleration waveform output from an acceleration sensor crosses a certain threshold, and the integral of the amplitude of the acceleration waveform, and records them as time series information.

A sleep state determination instrument including a vibration detection section that detects a vibration of a sleeper on a bed, and an activity amount computing section that computes the amount of activity of the sleeper per sampling unit time on the basis of the vibration detected by the vibration detection section is also known. The sleep state determination instrument includes a sleep determination value computing section that computes the total sum of values obtained by multiplying the activity amount at the current time and the activity amount computed before the current time by a correction factor weighted in accordance with time, as a sleep determination value. The sleep state determination instrument also includes a sleep state determination section that determines that the sleep state is sleeping if the sleep determination value exceeds a certain threshold and otherwise determines that the sleep state is awake.

It is known that arithmetic processing of a measuring device that is mounted on a subject and performs a plurality of kinds of calculations on measurement data is changed by remote control. A measurement terminal includes a sensor that generates measurement data on the movement or health condition of a patient, and a communication section that receives, from a setting terminal, an arithmetic instruction indicating at least one kind of calculation among a plurality of kinds of preset calculations. The measurement terminal includes a calculation section that may output results of the plurality of kinds of calculations by performing the plurality of kinds of calculations on the measurement data generated by the sensor and that performs the calculation indicated by the arithmetic instruction on the measurement data.

Examples of the related art include Japanese National Publication of International Patent Application No. 2009-538720, Japanese Laid-open Patent Publication No. 2000-245713, Japanese Laid-open Patent Publication No. 5-95934, Japanese Laid-open Patent Publication No. 2010-227191, Japanese Laid-open Patent Publication No. 2005-198829, Japanese Laid-open Patent Publication No. 2007-75428, Japanese Laid-open Patent Publication No. 2008-283, Japanese Laid-open Patent Publication No. 2010-264193 and Japanese Laid-open Patent Publication No. 2011-142966.

Akihisa Moriya et al., “Sleep state estimation method using pulse wave and its application” Journal of Human Interface Society, Vol. 10, No. 2, pages 207-214, 2008 is another example of the related art.

### SUMMARY

According to an aspect of the invention, an apparatus for determining depth of sleep includes a heart rate sensor configured to detect a heart rate of a subject in a non-contact manner, and a processor, wherein the processor performs, determining depth of sleep of the subject from the heart rate detected by the heart rate sensor, estimating a sleep cycle of the subject on the basis of a plurality of times at each of which a change in the depth of sleep occurs, and stopping the heart rate sensor, after completion of acquiring the plurality of times, at least until an anticipated time of a change in the depth of sleep.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 illustrates a first example of the hardware configuration of an electronic apparatus;

FIG. 2A illustrates a first example of the arrangement positions of sensors, and FIG. 2B illustrates an example of the use of the electronic apparatus;

FIG. 3 illustrates a second example of the arrangement positions of sensors;

FIG. 4 is a first example of a functional block diagram of the electronic apparatus;

FIG. 5A and FIG. 5B illustrate a first example and a second example of a time table, respectively, and FIG. 5C illustrates an example of a sleep cycle table;

FIG. 6 is a flowchart illustrating an example of operations of the electronic apparatus;

FIG. 7 illustrates a first example of a first determining operation;

FIG. 8 illustrates an example of a second determining operation;

FIG. 9 is a block diagram illustrating a second example of the hardware configuration of the electronic apparatus;

FIG. 10 is a second example of the functional block diagram of the electronic apparatus; and

FIG. 11 is a flowchart illustrating a second example of the first determining operation.

### DESCRIPTION OF EMBODIMENTS

Various techniques, such as a technique of measuring a heart rate by acquiring variations in position of a subject's skin surface by using high frequency electromagnetic waves such as a millimeter wave to the terahertz band (30 GHz to 30 THz), are utilized as approaches for measuring a heart rate in a non-contact manner. Determining depth of sleep from a heart rate measured in a non-contact manner may reduce the load imposed on a subject.

An apparatus for determining depth of sleep (referred to also as a “sleep depth determining apparatus”) is considered to be continuously used while a subject is sleeping. Accordingly, the sleep depth determining apparatus may be used for a comparatively long time. One of objects of the disclosed apparatus and method is to reduce the power consumption of a sleep depth determining apparatus that determines depth of sleep from a heart rate measured in a non-contact manner.

### 1. First Embodiment

### 1.1. First Example of Hardware Configuration of Electronic Apparatus

Hereinafter, preferred embodiments of an electronic apparatus for implementing an apparatus for determining depth of sleep will be described with reference to the accompanying drawings. The electronic apparatus may be a portable electronic apparatus that a user may carry, for example. The electronic apparatus may be a personal digital assistant, a mobile phone, a portable music player, a game device, a portable personal computer, a digital still camera, a digital camcorder, portable television (TV) set, a portable navigation device, or the like.

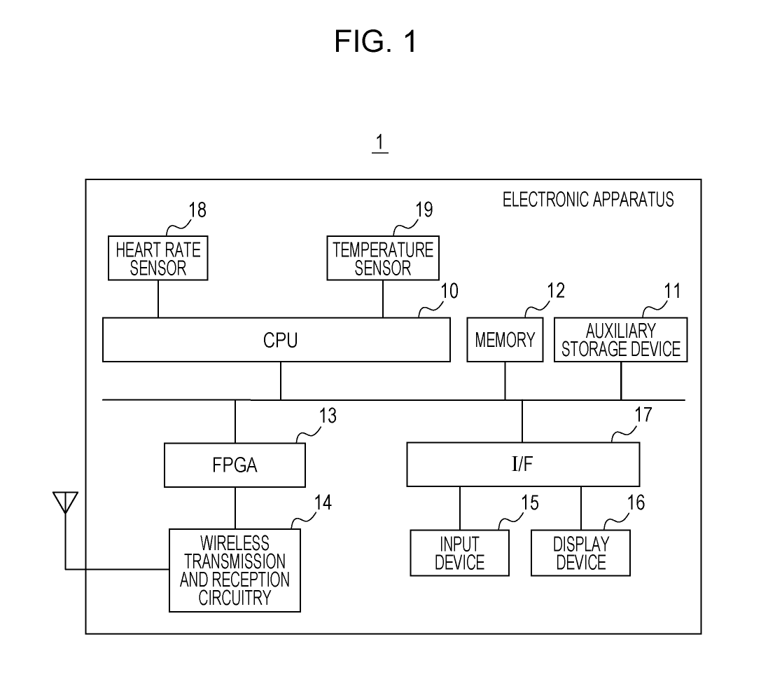

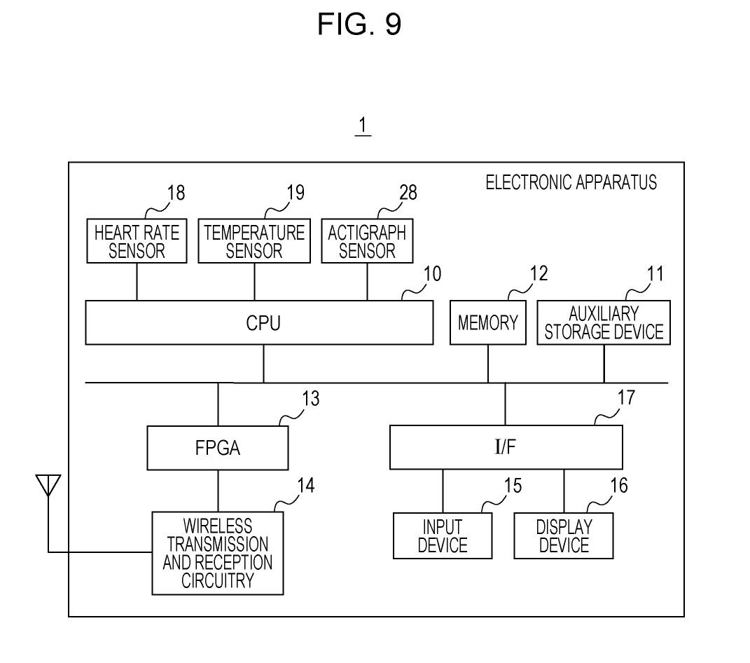

FIG. 1 illustrates a first example of the hardware configuration of the electronic apparatus. The hardware configuration illustrated in FIG. 1 is one of examples of the hardware configuration for implementing an electronic apparatus 1. Any other hardware configuration may be employed when it allows a process of determining depth of sleep described herein to be performed. The same applies to a second example of the hardware configuration illustrated in FIG. 9.

The electronic apparatus 1 includes a central processing unit (CPU) 10, an auxiliary storage device 11, a memory 12, a field-programming gate array (FPGA) 13, wireless transmission and reception circuitry 14, an input device 15, a display device 16, an interface circuit 17, a heart rate sensor 18, and a temperature sensor 19. Note that an interface circuit is indicated as “I/F” in the accompanying drawings.

The CPU 10 executes a computer program stored in the auxiliary storage device 11 to perform information processing of application software being used by a user of the electronic apparatus 1. The CPU 10 also executes a computer program stored in the auxiliary storage device 11 to perform a process of determining depth of sleep (hereinafter referred to as a “sleep depth determining process”), which will be described below. The auxiliary storage device 11 includes a nonvolatile storage device for storing a computer program and data used for execution of the computer program. The nonvolatile storage device may be a flash memory, a hard disk, or read-only memory (ROM), for example.

The memory 12 stores data that is used when the CPU 10 executes a computer program and temporary data. The memory 12 may contain a random access memory (RAM). The FPGA 13 performs baseband signal processing of signals transmitted and received in a wireless communication manner by the wireless transmission and reception circuitry 14. In the case where the electronic apparatus 1 is a mobile station apparatus, the FPGA 13 may perform baseband signal processing of signals transmitted and received in a wireless communication manner to and from the base station apparatus. The electronic apparatus 1 of another embodiment may include a digital signal processor (DSP) that performs baseband signal processing, in place of the FPGA 13, and a memory in which firmware to be performed by the DSP is stored. The electronic apparatus 1 may include logic circuits, such as large scale integration (LSI) circuits and application specific integrated circuits (ASICs), for baseband processing in place of the FPGA 13.

The input device 15 is a user interface device for accepting an input operation of a user to the electronic apparatus 1. The input device 15 includes ten keys for inputting telephone numbers, cursor keys, and keys dedicated for particular operations of the electronic apparatus 1, for example. The input device 15 may include a touch panel. The display device 16 displays a user interface of application software that the CPU 10 executes. The display device 16 also displays the result of a determination in the sleep depth determining process performed using the CPU 10. The display devices 16 may be a liquid crystal display device, an organic light emitting diode, or a field emission display, for example. The interface circuit 17 performs signal processing for input and output of signals between the input device 15 and display device 16, and the processor 10.

The heart rate sensor 18 includes a Doppler radar that irradiates a subject with electromagnetic waves from a millimeter wave to a wave of a high frequency in the terahertz band (30 GHz to 30 THz), and measures a heart rate by acquiring variations in the position of a subject's skin surface. In another embodiment, the heart rate sensor 18 may record a face, for example, with a camera, and detect pulses from changes of the red, green, and blue (RGB) values of the moving picture. In another embodiment, the heart rate sensor 18 may measure pulses by extracting vascular dynamics continuously photographed with near-infrared rays.

The temperature sensor 19 is a sensor that may measure the body surface temperature of a subject. For example, the temperature sensors 19 may be a thermograph or an infrared array sensor. In the case of using the electronic apparatus 1 in bed, the temperature sensor 19 may be a bandgap temperature sensor, for example.

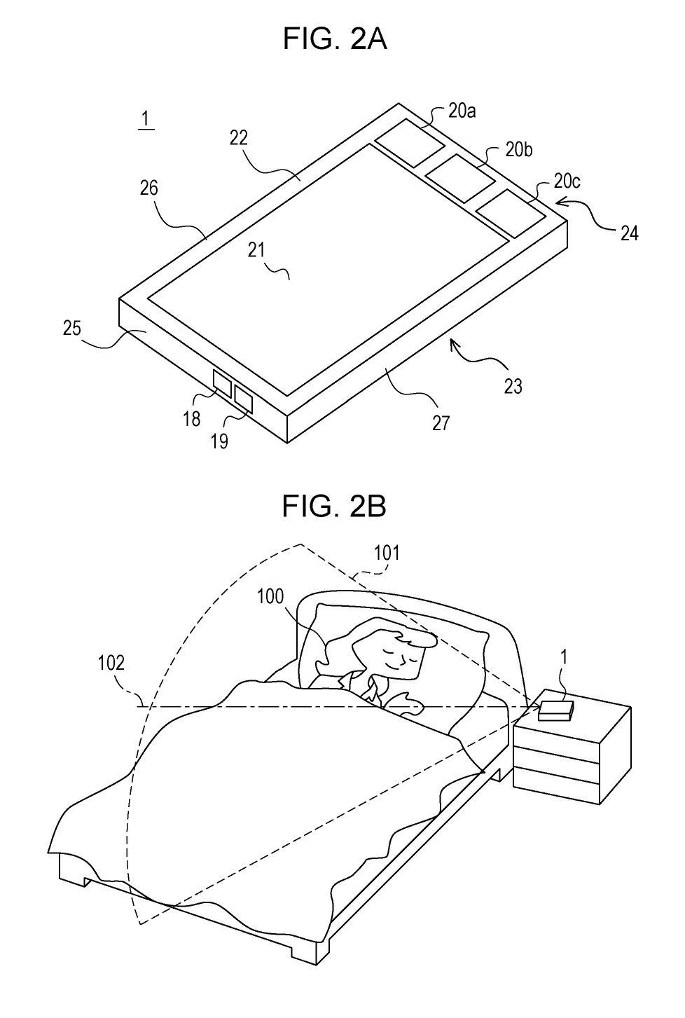

FIG. 2A illustrates a first example of the arrangement positions of the heart rate sensor 18 and the temperature sensor 19. Reference characters 20a to 20c denote dedicated keys for operations of the electronic apparatus 1, and reference character 21 denotes a touch panel indicator. The dedicated keys 20a to 20c and the touch panel indicator 21 are examples of the input device 15 and the display device 16. Reference character 22 denotes the front face of the electronic apparatus 1, reference character 23 denotes the back face of the electronic apparatus 1, reference character 24 denotes the bottom face of the electronic apparatus 1, reference character 25 denotes the top face of the electronic apparatus 1, and reference characters 26 and 27 denote side faces of the electronic apparatus 1. As illustrated, the heart rate sensor 18 and the temperature sensor 19 may be provided in the top face 25 of the electronic apparatus 1. In other embodiments, the heart rate sensor 18 and the temperature sensor 19 may be provided in any of other faces 22 to 24, 26, and 27.

FIG. 2B illustrates an example of the use of the electronic apparatus 1. Reference character 100 denotes a subject, reference character 101 denotes an example of the measuring range of the heart rate sensor 18 and temperature sensor 19, and reference character 102 denotes the central axis of the measuring range of the heart rate sensor 18 and temperature sensor 19. The electronic apparatus 1 is used while being placed on a stand close to the head of a bed, for example, and the electronic apparatus 1 is placed at a height of from −10 cm to +25 cm from the bed. Note that the electronic apparatus 1 may be used on the bed.

The electronic apparatus 1 is placed so that a central axis 102 of the measuring range of the heart rate sensor 18 points below the position of the jaw of a subject. For example, the face of the electronic apparatus 1 on which the heart rate sensor 18 is formed is turned below the position of the jaw of the subject.

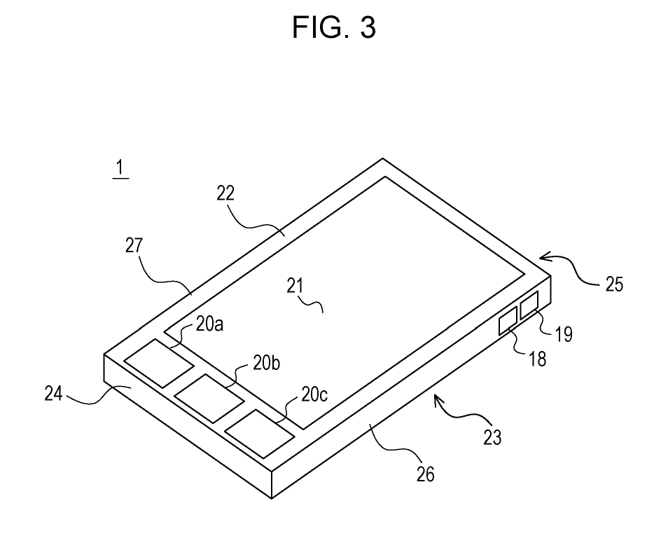

FIG. 3 illustrates a second example of the arrangement positions of sensors. The heart rate sensor 18 and the temperature sensor 19 may be provided in the side face 26 of the electronic apparatus 1 so that the measuring range of the heart rate sensor 18 and temperature sensor 19 is positioned to the side of the electronic apparatus 1 when the electronic apparatus 1 is held in a cradle in such a manner that the bottom face 24 points down. The heart rate sensor 18 and the temperature sensor 19 may be provided in the other side face 27, the front face 22, or the back face 23.

### 1.2. First Example of Functional Configuration of Electronic Apparatus

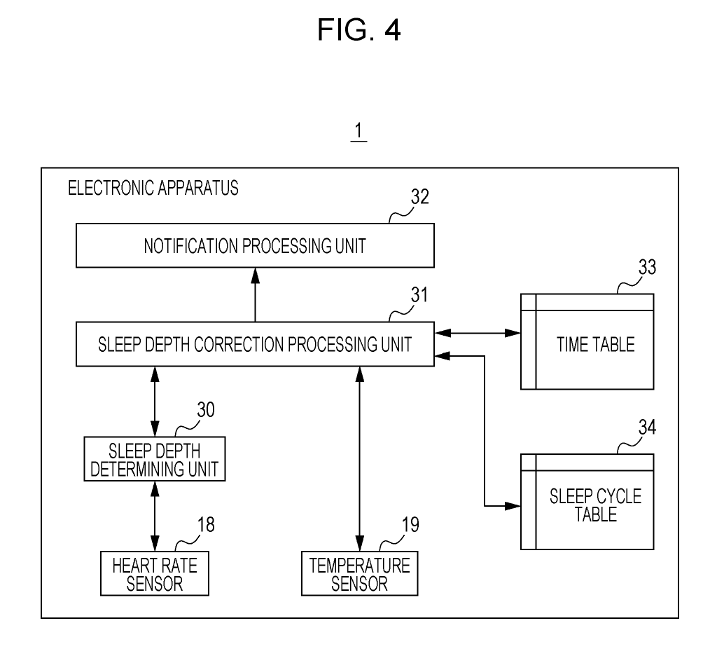

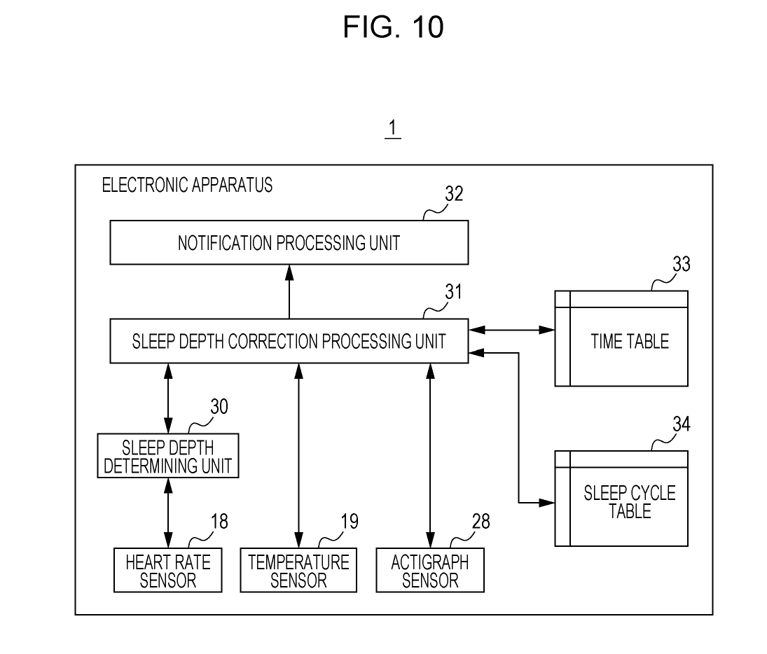

Functions of the electronic apparatus 1 implemented using the above hardware configuration will be described next. FIG. 4 is a first example of a functional block diagram of the electronic apparatus 1. The electronic apparatus 1 includes the heart rate sensor 18, the temperature sensor 19, a sleep depth determining unit 30, a sleep depth correction processing unit 31, a notification processing unit 32, a time table 33, and a sleep cycle table 34. Operations of the sleep depth determination unit 30, the sleep depth correction processing unit 31, and the notification processing unit 32 are performed by the CPU 10 illustrated in FIG. 1. The time table 33 and the sleep cycle table 34 are stored in the auxiliary storage device 11 or the memory 12. Note that FIG. 4 is illustrated with a central focus on the functions related to the following description. The electronic apparatus 1 may include components other than those illustrated in the drawing. The same applies to a second example of the functional block diagram illustrated in FIG. 10.

The sleep depth determining unit 30 determines depth of sleep from the ratio of a low frequency component to a high frequency component of the fluctuation in a heart rate detected by the heart rate sensor 18. The sleep depth correction processing unit 31 outputs, to the notification processing unit 32, sleep depth information representing depth of sleep that has been determined on the basis of the result of the determination made by the sleep depth determining unit 30.

In accordance with the result of the determination made by the sleep depth determining unit 30, the sleep depth correction processing unit 31 stores, in the time table 33, a time at which a shift to relatively deep sleep occurs, a time at which a shift from the relatively deep sleep to relatively light sleep occurs, and a time at which a shift from the relatively light sleep to relatively deep sleep occurs. Regarding the types and number of times at which changes in depth of sleep occur, any other types of times and any other number of times that are sufficient to estimate the sleep cycle of a subject may be stored in the time table 33 by the sleep depth correction processing unit 31.

The term “sleep cycle” as used herein refers to a first period P1 from a time at which a shift to relatively light sleep occurs to a time at which a shift to relatively deep sleep occurs, a second period P2 from the time at which a shift to relatively deep sleep occurs to a time at which a shift to relatively light sleep occurs, or the period of 1 cycle in which these periods are combined together. The sleep depth correction processing unit 31 may store, in the time table 33, a time at which a shift to relatively light sleep occurs, a time at which a shift from relatively light sleep to relatively deep sleep occurs, and a time at which a shift from the relatively deep sleep to relatively light sleep occurs.

The sleep depth correction processing unit 31 may regard relatively deep non-rapid eye movement (NREM or non-REM) sleep as relatively deep sleep. The sleep depth correction processing unit 31 may regard relatively light NREM sleep or rapid eye movement (REM) sleep as relatively light sleep. The sleep depth correction processing unit 31 stores the times at which a subject shifts to relatively deep NREM sleep, to the subsequent relatively light NREM sleep or REM sleep, and to the subsequent relatively deep NREM sleep, respectively, in the time table 33. For example, the sleep depth correction processing unit 31 stores the times at which a subject shifts to first relatively deep NREM sleep, to first relatively light NREM sleep or REM sleep, and to second relatively deep NREM sleep, respectively, in the time table 33.

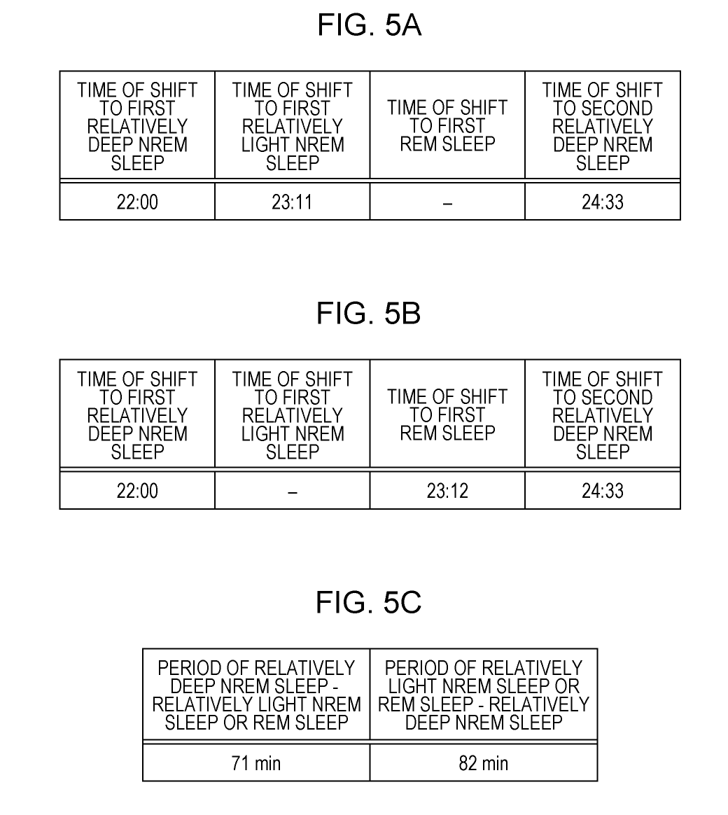

In the time table 33, there are provided areas where times at which shifts to relatively deep sleep occur and times at which shifts to relatively light sleep occur are to be stored. FIG. 5A illustrates a first example of the time table 33. In the time table 33, for example, there are provided areas where times at which shifts to first relatively deep NREM sleep, to first relatively light NREM sleep, to first REM sleep, and to second relatively deep NREM sleep occur are to be stored. FIG. 5A illustrates a state in which times “22:00”, “23:11”, and “24:33” at which shifts to first relatively deep NREM sleep, first relatively light NREM sleep, and second relatively deep NREM sleep occur, respectively, are stored in the time table 33.

FIG. 5B illustrates a state in which times “22:00”, “23:12”, and “24:33” at which shifts to first relatively deep NREM sleep, first REM sleep, and second relatively deep NREM sleep occur, respectively, are stored in the time table 33. In another embodiment, the time at which a shift to first relatively light NREM sleep occurs and the time at which a shift to REM sleep occurs may share the same storage area.

The sleep depth correction processing unit 31 estimates the sleep cycle of the subject from time information stored in the time table 33. The sleep depth correction processing unit 31 stores the estimated first period P1 and second period P2 in the sleep cycle table 34. The first period P1 is a period from a time at which a shift to relatively light sleep occurs to a time at which a shift to relatively deep sleep occurs, and the second period P2 is a period from the time at which a shift to relatively deep sleep occurs to a time at which a shift to relatively light sleep occurs.

FIG. 5C illustrates an example of the sleep cycle table 34. FIG. 5C indicates that the estimated second period P2, which is a period from a time at which a shift to relatively deep NREM sleep occurs to a time at which a shift to relatively light NREM sleep or REM sleep occurs, is “71 min (minutes)”. FIG. 5C indicates that the estimated first period P1, which is a period from the time at which a shift to relatively light NREM sleep or REM sleep occurs to a time at which a shift to relatively deep NREM sleep occurs is “82 min”.

After completion of collection of time data used for estimation of the sleep cycle, that is, estimation of the first period P1 and the second period P2, the sleep depth correction processing unit 31 stops a determination being made by the sleep depth determining unit 30 and stops the heart rate sensor 18.

After estimating the sleep cycle, the sleep depth correction processing unit 31 acquires information on the subject's temperature from the temperature sensor 19 at an anticipated time at which the next change in depth of sleep will occur. At each anticipated time, the sleep depth correction processing unit 31 determines, on the basis of a change in the subject's temperature after the anticipated time, whether there is a change in depth of sleep of the subject, that is, a shift from relatively light sleep to relatively deep sleep or a shift from relatively deep sleep to relatively light sleep.

The sleep depth correction processing unit 31 outputs, to the notification processing unit 32, sleep depth information representing the subject's depth of sleep that has been determined on the basis of the estimated sleep cycle and a change in temperature. Note that the temperature sensor 19 may continuously operate during a measurement of depth of sleep. Alternatively, after detecting a change in depth of sleep, the sleep depth correction processing unit 31 may turn off the temperature sensor 19 until an anticipated time of the next change in depth of sleep is reached.

In another embodiment, after estimating the sleep cycle, the sleep depth correction processing unit 31 may activate the heart rate sensor 18 at an anticipated time at which the next change in depth of sleep will occur, and may determine depth of sleep in accordance with the result of a determination made by the sleep depth determining unit 30. In this case, after detecting the change in depth of sleep, the sleep depth correction processing unit 31 may turn off the heart rate sensor 18 until an anticipated time of the next change in depth of sleep is reached.

The notification processing unit 32 outputs the sleep depth information from the sleep depth correction processing unit 31 to the display device 16.

### 1.3. First Example of Sleep Depth Determining Process

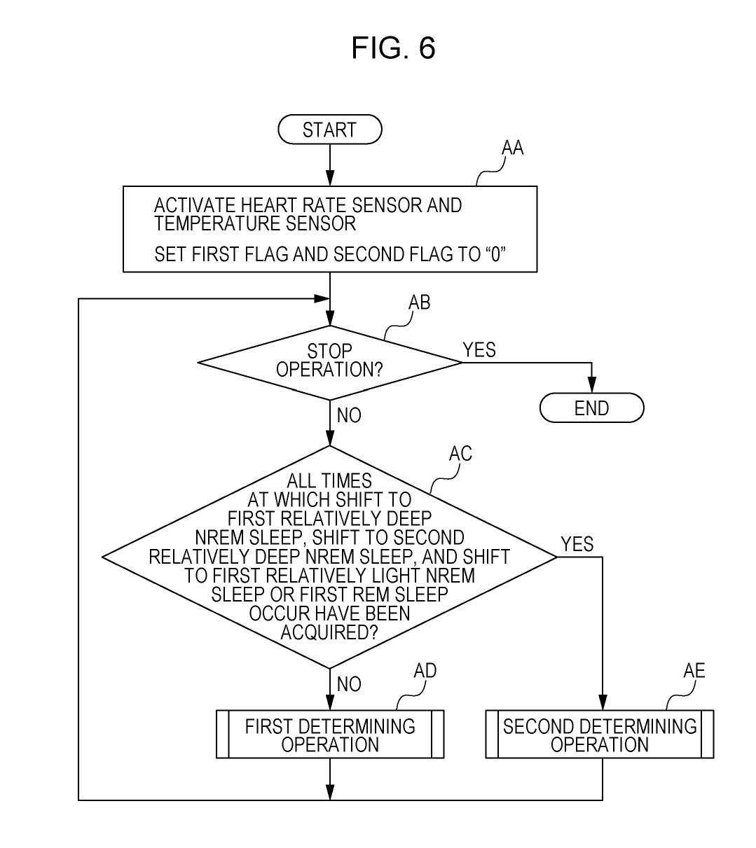

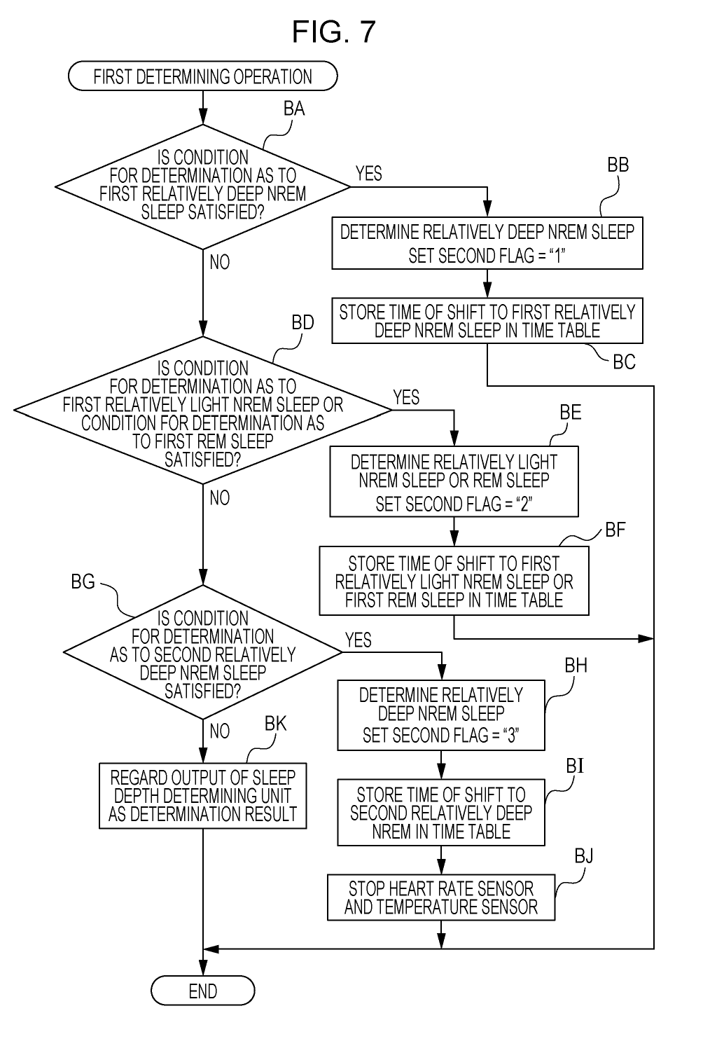

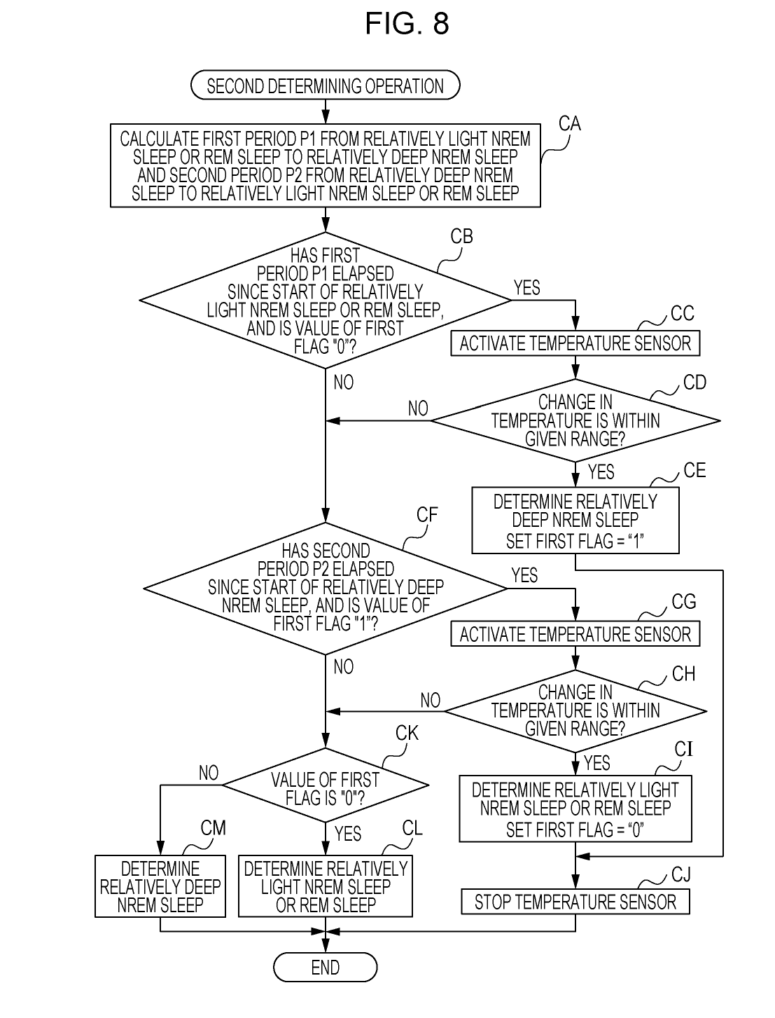

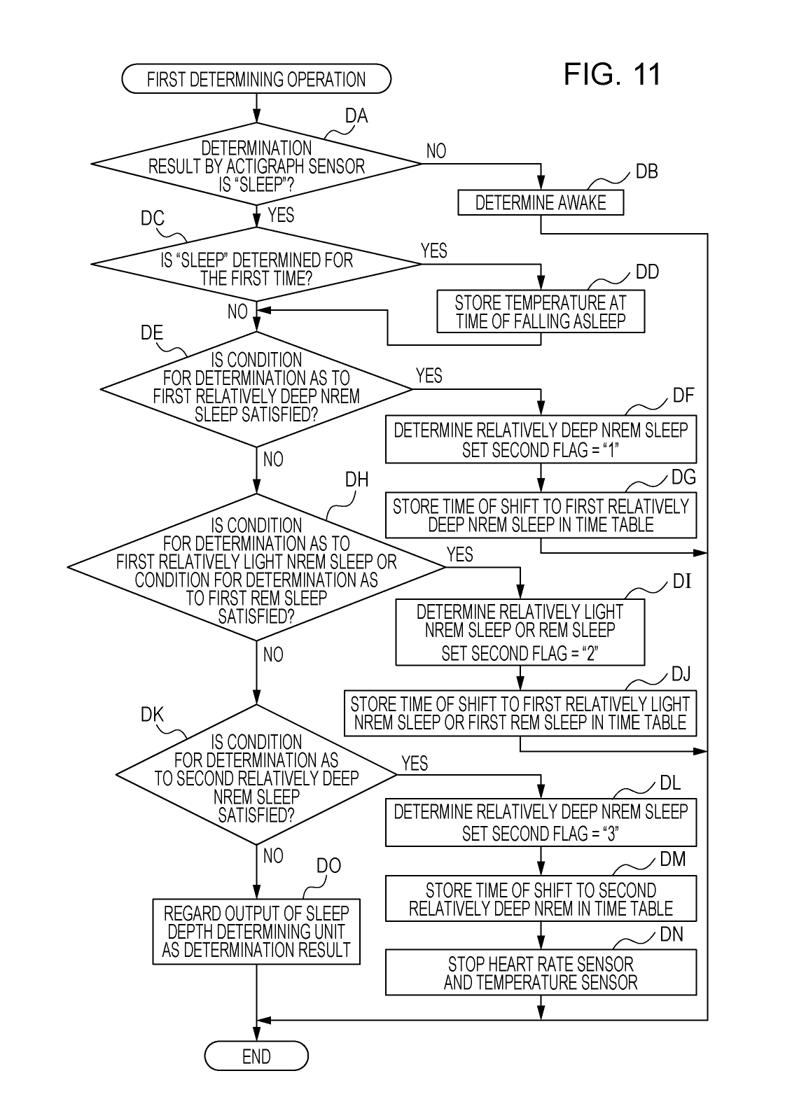

Subsequently, the sleep depth determining process performed by the electronic apparatus 1 will be described. FIG. 6 is a flowchart illustrating an example of operations of the electronic apparatus. Note that a series of operations that will be described with reference to FIG. 6 may be construed as a method including a plurality of procedures. In this case, “operation” may be read as “step”. The same applies to the cases of FIG. 7, FIG. 8, and FIG. 11.

In operation AA, the sleep depth correction processing unit 31 activates the heart rate sensor 18 and the temperature sensor 19. The CPU 10 sets the values of a first flag and a second flag, which are two flag variables, to “0”. The first flag is used for storing depth of sleep of the subject after the sleep cycle has been estimated. The value “0” indicates relatively light NREM sleep or REM sleep, and the value “1” indicates relatively deep NREM sleep. The second flag is used for storing depth of sleep of the subject before the sleep cycle is estimated. The value “0” indicates a state before a shift to first relatively deep NREM sleep, the value “1” indicates first relatively deep NREM sleep, the value “2” indicates first relatively light NREM sleep or REM sleep, and the value “3” indicates second relatively deep NREM sleep.

In operation AB, when the input device 15 accepts a stop operation (operation AB: Yes), then the sleep depth correction processing unit 31 completes the process. When the input device 15 does not accept a stop operation (operation AB: No), then the process proceeds to operation AC.

In operation AC, the sleep depth correction processing unit 31 determines whether acquisition of times at which changes in depth of sleep occur, which are used for estimation of a sleep cycle, has been completed. For example, the sleep depth correction processing unit 31 determines whether all the times at which a shift to first relatively deep NREM sleep, a shift to second relatively deep NREM sleep, and a shift to first relatively light NREM sleep or REM sleep occur have been acquired. When all the change times have been acquired (operation AC: Yes), then the process proceeds to operation AE. When there remains a change time that has not been acquired (operation AC: No), then the process proceeds to operation AD.

In operation AD, the sleep depth correction processing unit 31 performs a first determining operation. The first determining operation will be described later with reference to FIG. 7. Then, the process returns to operation AB. In operation AE, the sleep depth correction processing unit 31 performs a second determining operation. The second determining operation will be described later with reference to FIG. 8. Then, the process returns to operation AB.

Subsequently, the first determining operation in operation AD of FIG. 6 will be described. FIG. 7 illustrates a first example of the first determining operation. In operation BA, the sleep depth correction processing unit 31 determines whether a condition for determining, on the basis of the result of a determination made by the sleep depth determining unit 30, whether the subject has shifted to first relatively deep NREM sleep is satisfied. This determination condition may be a condition that the value of the second flag is “0” and the result of a determination made by the sleep depth determining unit 30 is relatively deep NREM sleep, for example. When the determination condition is satisfied (operation BA: Yes), then the process proceeds to operation BB. When the determination condition is not satisfied (operation BA: No), then the process proceeds to operation BD.

In operation BB, the sleep depth correction processing unit 31 determines that the subject has shifted to relatively deep NREM sleep, and outputs sleep depth information to the notification processing unit 32. Also, the sleep depth correction processing unit 31 changes the value of the second flag to “1”. In operation BC, the sleep depth correction processing unit 31 stores the current time, as a time at which a shift to first relatively deep NREM sleep occurs, in the time table 33. Then, the first determining operation is completed.

In operation BD, the sleep depth correction processing unit 31 determines whether a condition for determining, on the basis of the result of a determination made by the sleep depth determining unit 30, whether the subject has shifted to first relatively light NREM sleep or REM sleep is satisfied. This determination condition may be a condition that satisfies all the following three sub-conditions (1) to (3).

(1) The value of the second flag is “1”.

(2) Sixty minutes have not yet elapsed since the shift to first deep NREM sleep.

(3) The result of a determination made by the sleep depth determining unit 30 is relatively light NREM sleep or REM sleep.

When the determination condition is satisfied (operation BD: Yes), then the process proceeds to operation BE. When the determination condition is not satisfied (operation BD: No), then the process proceeds to operation BG.

In operation BE, the sleep depth correction processing unit 31 determines that the subject has shifted to relatively light NREM sleep or REM sleep, and outputs sleep depth information to the notification processing unit 32. Also, the sleep depth correction processing unit 31 changes the value of the second flag to “2”. In operation BF, the sleep depth correction processing unit 31 stores the current time, as a time at which a shift to first relatively light NREM sleep or first REM sleep occurs, in the time table 33. Then, the first determining operation is completed.

In operation BG, the sleep depth correction processing unit 31 determines whether a condition for determining, on the basis of the result of a determination made by the sleep depth determining unit 30, whether the subject has shifted to second relatively deep NREM sleep is satisfied. This determination condition may be a condition that satisfies all the following three sub-conditions (11) to (13).

(11) The value of the second flag is “2”.

(12) Sixty minutes have not yet elapsed since the shift to first light NREM sleep or first REM sleep.

(13) The result of a determination made by the sleep depth determining unit 30 is relatively deep NREM sleep.

When the determination condition is satisfied (operation BG: Yes), then the process proceeds to operation BH. When the determination condition is not satisfied (operation BG: No), then the process proceeds to operation BK.

In operation BH, the sleep depth correction processing unit 31 determines that the subject has shifted to relatively deep NREM sleep, and outputs sleep depth information to the notification processing unit 32. Also, the sleep depth correction processing unit 31 changes the value of the second flag to “3”. In operation BI, the sleep depth correction processing unit 31 stores the current time, as a time at which a shift to second relatively deep NREM sleep occurs, in the time table 33. In operation BJ, the sleep depth correction processing unit 31 stops the heart rate sensor 18 and the temperature sensor 19. Then, the first determining operation is completed.

In operation BK, the sleep depth correction processing unit 31 regards the output of the sleep depth determining unit 30, just as it is, as the result of the determination as to depth of sleep, and outputs sleep depth information to the notification processing unit 32. Then, the first determining operation is completed.

Subsequently, the second determining operation in operation AE of FIG. 6 will be described. FIG. 8 illustrates an example of the second determining operation. In operation CA, the sleep depth correction processing unit 31 calculates the “sleep cycle” in accordance with time information of the time table 33. That is, the sleep depth correction processing unit 31 calculates the first period P1 obtained by estimating a time period from the time at which a shift to relatively light NREM sleep or REM sleep occurs to the time at which a shift to relatively deep NREM sleep occurs. The sleep depth correction processing unit 31 calculates the second period P2 obtained by estimating a time period from the time at which a shift to relatively deep NREM sleep occurs to the time at which a shift to relatively light NREM sleep or REM sleep occurs. The sleep depth correction processing unit 31 stores the first period P1 and the second period P2 in the sleep cycle table 34. Calculation of the “sleep cycle” may be performed only once.

In operation CB, the sleep depth correction processing unit 31 determines whether the first period P1 has elapsed since the start of relatively light NREM sleep or REM sleep and whether the value of the first flag is “0”. When the first period P1 has elapsed since the start of relatively light NREM sleep or REM sleep, and the value of the first flag is “0” (operation CB: Yes), then the process proceeds to operation CC, whereas when not (operation CB: No), then the process proceeds to operation CF.

In operation CC, the sleep depth correction processing unit 31 activates the temperature sensor 19. In operation CD, the sleep depth correction processing unit 31 determines whether a change in temperature of the subject compared to the temperature measured upon the previous change in depth of sleep is within a certain range. The certain range may be a range from 2.5 to 3.5° C., for example. When the temperature change is within the certain range (operation CD: Yes), then the process proceeds to operation CE. When the temperature change is not within the certain range (operation CD: No), then the process proceeds to operation CF.

In operation CE, the sleep depth correction processing unit 31 determines that the subject has shifted to relatively deep NREM sleep, and outputs sleep depth information to the notification processing unit 32. Also, the sleep depth correction processing unit 31 changes the value of the first flag to “1”.

Then, the process proceeds to operation CJ.

In operation CF, the sleep depth correction processing unit 31 determines whether the second period P2 has elapsed since the start of relatively deep NREM sleep and whether the value of the first flag is “1”. When the first period P2 has elapsed since the start of relatively deep NREM sleep, and the value of the first flag is “1” (operation CF: Yes), then the process proceeds to operation CG, whereas when not (operation CF: No), then the process proceeds to operation CK.

In operation CG, the sleep depth correction processing unit 31 activates the temperature sensor 19. In operation CH, the sleep depth correction processing unit 31 determines whether a change in temperature of the subject compared to the temperature measured upon the previous change in depth of sleep is within the certain range. When the temperature change is within the certain range (operation CH: Yes), then the process proceeds to operation CI. When the temperature change is not within the certain range (operation CH: No), then the process proceeds to operation CK.

In operation CI, the sleep depth correction processing unit 31 determines that the subject has shifted to relatively light NREM sleep or REM sleep, and outputs sleep depth information to the notification processing unit 32. Also, the sleep depth correction processing unit 31 changes the value of the first flag to “0”. Then, the process proceeds to operation CJ.

In operation CJ, the sleep depth correction processing unit 31 stops the temperature sensor 19. Then, the second determining operation is completed. In operation CK, the sleep depth correction processing unit 31 determines whether the value of the first flag is “0”. When the value of the first flag is “0” (operation CK: Yes), then the process proceeds to operation CL. When the value of the first flag is not “0” (operation CK: No), then the process proceeds to operation CM.

In operation CL, the sleep depth correction processing unit 31 determines that the subject has shifted to relatively light NREM sleep or REM sleep, and outputs sleep depth information to the notification processing unit 32. Then, the second determining operation is completed. In operation CM, the sleep depth correction processing unit 31 determines that the subject has shifted to relatively deep NREM sleep, and outputs sleep depth information to the notification processing unit 32. Then, the second determining operation is completed.

### 1.4. Effects

According to this embodiment, since depth of sleep is determined from the heart rate measured in a non-contact manner, the load imposed on the subject at the time of determining depth of sleep may be made small. Since the heart rate sensor that detects a heart rate in a non-contact manner is stopped after information on times at which shifts in depth of sleep occur, which are used for estimation of a sleep cycle, has been collected, power consumption may be reduced. Accordingly, even regarding a portable electronic apparatus whose battery capacity is limited, the above-described sleep depth determining process may be performed by using a heart rate sensor with comparatively large power consumption, such as a heart rate sensor that uses high-frequency electromagnetic waves.

According to this embodiment, after estimating the sleep cycle, the sleep depth correction processing unit 31 determines depth of sleep of the subject on the basis of the estimated sleep cycle and a change in temperature of the subject. Accordingly, even if there is a change in sleep cycle of the subject while the subject is sleeping, the presence of a change in depth of sleep may be more accurately determined by correcting the anticipated time of a change in depth of sleep using the result of a detection made by a temperature sensor with comparatively small power consumption. A wrong detection caused by a change in temperature independent of a change in depth of sleep may be inhibited. Thus, it may become possible to determine depth of sleep with high accuracy without using a heart rate sensor with comparatively large power consumption.

### 2. Second Embodiment

Subsequently, another embodiment will be described. In this embodiment, the sleep depth correction processing unit 31 determines a change in depth of sleep by combining the result of a determination made by the sleep depth determining unit 30 with a change in the subject's temperature measured by the temperature sensor 19 in the first determining operation described in “1.3. First Example of Sleep Depth Determining Process”.

For example, in operation BD, the sleep depth correction processing unit 31 determines whether a condition for determining, on the basis of the result of a determination made by the sleep depth determining unit 30 and a change in temperature of the subject, whether the subject has shifted to first relatively light NREM sleep or REM sleep is satisfied. This determination condition may be a condition that satisfies all the following four sub-conditions (1) to (4).

(1) The value of the second flag is “1”.

(2) Sixty minutes have not yet elapsed since a shift to first deep NREM sleep.

(3) The result of a determination made by the sleep depth determining unit 30 is relatively light NREM sleep or REM sleep.

(4) Compared to temperature of the subject at the time when relatively deep NREM sleep is determined in operation BB, the temperature increases by a certain number of degrees or more. The certain number of degrees may be 0.3° C., for example.

Also, for example, in operation BG, the sleep depth correction processing unit 31 determines whether a condition for determining, on the basis of the result of a determination made by the sleep depth determining unit 30 and a change in temperature of the subject, whether the subject has shifted to second relatively deep NREM sleep is satisfied. This determination condition may be a condition that satisfies all the following four sub-conditions (11) to (14).

(11) The value of the second flag is “2”.

(12) Sixty minutes have not yet elapsed since a shift to first light NREM sleep or first REM sleep.

(13) The result of a determination made by the sleep depth determining unit 30 is relatively deep NREM sleep.

(14) Compared to temperature of the subject at the time when relatively light NREM sleep or REM sleep is determined in operation BE, the temperature decreases by a certain number of degrees or more. The certain number of degrees may be 0.3° C., for example.

According to this embodiment, the error of measurement of the heart rate sensor 18 is corrected by using a change in temperature of the subject, and thus it may become possible to improve accuracy in measurement. Note that the second embodiment may be combined with a third embodiment described below.

### 3. Third Embodiment

Subsequently, another embodiment will be described. In this embodiment, the electronic device 1 includes an actigraph sensor that detects body movement of the subject. In this embodiment, the sleep depth correction processing unit 31 determines a change in depth of sleep by combining the result of a determination made by the sleep depth determining unit 30 in the first determining operation described in “1.3. First Example of Sleep Depth Determining Process”, a change in temperature of the subject, and the body movement detected by the actigraph sensor.

### 3.1. Second Example of Configuration of Electronic Apparatus

FIG. 9 and FIG. 10 are a block diagram illustrating the second example of the hardware configuration of the electronic apparatus 1 and the second example of the functional block diagram thereof. Regarding the same operations as those of elements described with reference to FIG. 1 and FIG. 4, the description thereof is omitted. The electronic apparatus 1 includes an actigraph sensor 28 that detects body movement of the subject.

The actigraph sensor 28 may be a non-contact sensor to determine a change in the distance to the subject that is caused to occur by body movement of the subject, for example. In this case, the actigraph sensor 28 may be an ultrasonic sensor or a photosensor, for example. The actigraph sensor 28 may be an acceleration sensor to detect acceleration that is caused by the subject's body movement and is applied to the sensor, for example.

In the case where the actigraph sensor 28 is a non-contact sensor, the actigraph sensor 28 may be provided in the same face as the face where the heart rate sensor 18 and the temperature sensor 19 are disposed, among the external faces 22 to 27 of the electronic apparatus 1 of FIG. 2A and of FIG. 3. In the case where the actigraph sensor 28 is an acceleration sensor, the actigraph sensor 28 may be provided in the inside of the electronic apparatus 1. For example, the actigraph sensor 28 may be provided in an electronic circuit board provided on the back face of the touch-panel indicator 21.

The sleep depth correction processing unit 31 determines, on the basis of the frequency of occurrence of the subject's body movement detected by the actigraph sensor 28, whether the subject is in a wake state or in a sleep state. When the subject is in a sleep state, then the sleep depth correction processing unit 31 determines whether a shift to relatively deep sleep after falling asleep, a shift from relatively deep sleep to relatively light sleep, and a shift from relatively light sleep to relatively deep sleep occur.

### 3.2. Second Example of Sleep Depth Determining Process

Subsequently, operations of the electronic apparatus 1 of a third embodiment will be described. Operations of the sleep depth determining process and the second determining operation may be the same as the operations of FIG. 6 and FIG. 8. FIG. 11 is a flowchart illustrating a second example of the first determining operation.

In operation DA, the sleep depth correction processing unit 31 determines, on the basis of the frequency of occurrence of the subject's body movement detected by the actigraph sensor 28, whether the subject is in a sleep state. When the subject is in a sleep state (operation DA: Yes), then the process proceeds to operation DC. When the subject is not in a sleep state (operation DA: No), then the process proceeds to operation DB. In operation DB, the sleep depth correction processing unit 31 determines that the subject is in a wake state, and outputs sleep depth information to the notification processing unit 32. Then, the first determining operation is completed.

In operation DC, the sleep depth correction processing unit 31 determines whether the result of a determination in operation DA is the first detection of the sleep state after the start of the sleep depth determining process. When the result of a determination in operation DA is the first detection of the sleep state (operation DC: Yes), then the process proceeds to operation DD, whereas when not (operation DC: No), then the process proceeds to operation DE. In operation DD, the sleep depth correction processing unit 31 determines that the subject has fallen asleep at the current time, and stores the subject's temperature detected by the temperature sensor 19, as a temperature at the time of falling asleep, in the memory 12.

In operation DE, the sleep depth correction processing unit 31 determines whether a condition for determining whether the subject has shifted to first relatively deep NREM sleep is satisfied. This determination condition may be a condition that satisfies all the following three sub-conditions (21) to (23).

(21) The value of the second flag is “0”.

(22) The result of a determination made by the sleep depth determining unit 30 is relatively deep NREM sleep.

(23) The temperature of the subject detected by the temperature sensor 19 decreases compared to that at the time of falling asleep by a certain number of degrees or more. The certain number of degrees may be 0.3° C., for example.

When the determination condition is satisfied (operation DE: Yes), then the process proceeds to operation DF. When the determination condition is not satisfied (operation DE: No), then the process proceeds to operation DH. The operation of operation DF and operation DG is the same as that of operation BB and operation BC of FIG. 7.

In operation DH, in accordance with the above-described sub-conditions (1) to (4) of “2. Second Embodiment”, the sleep depth correction processing unit 31 determines whether the subject has shifted to first relatively light NREM sleep or REM sleep. When the sub-conditions (1) to (4) are satisfied (operation DH: Y), then the process proceeds to operation DI. When the determination condition is not satisfied (operation DH: No), then the process proceeds to operation DK. The operation of operation DI and operation DJ is the same as that of operation BE and operation BF of FIG. 7.

In operation DK, in accordance with the above-described sub-conditions (11) to (14) of “2. Second Embodiment”, the sleep depth correction processing unit 31 determines whether the subject has shifted to second relatively deep NREM sleep. When the sub-conditions (11) to (14) are satisfied (operation DK: Y), then the process proceeds to operation DL. When the determination condition is not satisfied (operation DK: No), then the process proceeds to operation DO. The operation of operation DL to operation DO is the same as that of operation BH to operation BK of FIG. 7.

### 3.3. Effects

According to this embodiment, the error of measurement of the heart rate sensor 18 may be corrected by using the result of a detection of the subject's body movement made by the actigraph sensor 28 in addition to a change in the subject's temperature. The accuracy in measurement therefore may improve. In an embodiment, the sleep depth correction processing unit 31 may stop the heart rate sensor 18 and the temperature sensor 19 until a sleep state is detected from the result of a detection of the subject's body movement made by the actigraph sensor 28. The stop of the sensors allows power consumption to be reduced. In another embodiment, the heart rate sensor 18 using high-frequency electromagnetic waves may be used in addition to or in place of the actigraph sensor 28, so that body movement of a subject is detected and the detection is used for a determination of the sleep state of the subject.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An apparatus for determining depth of sleep, comprising:
a heart rate sensor configured to detect a heart rate of a subject in a non-contact manner; and
a processor,
wherein the processor performs,
determining depth of sleep of the subject from the heart rate detected by the heart rate sensor,
estimating a sleep cycle of the subject on the basis of a plurality of times at each of which a change in the depth of sleep occurs, and
stopping the heart rate sensor, after completion of acquiring the plurality of times, at least until an anticipated time of a change in the depth of sleep.

2. The apparatus according to claim 1, further comprising a temperature sensor, wherein the processor performs detecting the change in depth of sleep of the subject on the basis of a change in temperature of the subject detected by the temperature sensor at the anticipated time of the change in depth of sleep after estimating the sleep cycle.

3. The apparatus according to claim 2, wherein, after detecting the change in depth of the subject, the processor turns off the temperature sensor until the anticipated time of the next change in depth of sleep is reached.

4. The apparatus according to claim 2, wherein, in the estimating of the sleep cycle, the processor performs correction of a result of determining the depth of sleep from the heart rate, on the basis of a change in temperature of the subject detected by the temperature sensor.

5. The apparatus according to claim 1, wherein the processor performs, activating the heart rate sensor at the anticipated time of the next change in depth of sleep, and
detecting a change in depth of sleep of the subject on the basis of a heart rate of the subject detected by the heart rate sensor at an anticipated time of a change in depth of sleep after estimating the sleep cycle.

6. The apparatus according to claim 5, wherein, after detecting the change in depth of sleep of the subject, the processor turns off the heart rate sensor until an anticipated time of a next change in depth of sleep is reached.

7. The apparatus according to claim 1, further comprising a temperature sensor,
wherein, on the basis of a change in temperature of the subject detected by the temperature sensor, the processor performs correction of a result of determining the depth of sleep from the heart rate.

8. The apparatus according to claim 5, further comprising a temperature sensor,
wherein, on the basis of a change in temperature of the subject detected by the temperature sensor, the processor performs correction of a result of determining the depth of sleep from the heart rate.

9. The apparatus according to claim 1, further comprising a sensor configured to detect body movement of the subject,
wherein the processor performs determining of the depth of sleep of the subject when a sleep state of the subject is detected on the basis of a frequency of the body movement detected by the sensor.

10. A method for determining depth of sleep, comprising:
detecting a heart rate of a subject by a heart rate sensor provided in an electronic apparatus, the heart rate sensor being configured to detect a heart rate in a non-contact manner; and
causing a processor included in the electronic apparatus to perform,
determining depth of sleep of the subject from the heart rate detected by the heart rate sensor,
estimating a sleep cycle of the subject on the basis of a plurality of times at each of which a change in the depth of sleep occurs, and
stopping the heart rate sensor, after completion of acquiring the plurality of times, at least until an anticipated time of a change in the depth of sleep.
